(19) United States
(12) Patent Application Publication
(10) Pub. No.: US 2008/0114451 A1
(43) Pub. Date: May 15, 2008

(54) RETENTION COATINGS FOR DELIVERY SYSTEMS

(76) Inventors: Sean M. Stucke, Farmington, MN (US); Kimberly K.M. Lindsoe, Savage, MN (US); Ralph A. Chappa, Prior Lake, MN (US); Dale G. Swan, St. Louis Park, MN (US)

Correspondence Address: SURMODICS, INC., 9924 WEST 74TH STREET, EDEN PRAIRIE, MN, 55344, US

(21) Appl. No.: 12/016,343

(22) Filed: Jan. 18, 2008

Related U.S. Application Data
(62) Division of application No. 10/231,631, filed on Aug. 30, 2002.

(51) Int. Cl. A61L 27/34; A61F 2/82; C09K 3/16

(52) U.S. Cl. 623/1.46; 427/2.25; 528/271

(57) ABSTRACT

A coating composition, in both its uncrosslinked and crosslinked forms, for use in increasing the static friction of a surface of a delivery system comprising a medical device having a surface in contact with the surface of a delivery component, the static friction of the surface being increased in an amount sufficient to substantially maintain the position of the medical device on the delivery component against forces asserted on the delivery system as it navigates through a vessel of the body. The delivery system may comprise a balloon catheter as the delivery component and a stent as the medical device. A composition includes a polyether monomer, such as an alkoxy poly(alkylene glycol), a carboxylic acid-containing monomer, such as (meth)acrylic acid, optionally a photoderivatized monomer, and a hydrophilic monomer such as (meth)acrylamide.

### TECHNICAL FIELD

In one aspect, the present invention relates to hydrogel matrix coatings for a medical device system such as an intravascular stent deployment system. In another aspect, the invention relates to methods of using such hydrogel matrix coatings on a surface of a delivery system to increase the static friction of the surface of such delivery system.

### BACKGROUND OF THE INVENTION

Medical devices adapted to be used for intrusion into body cavities canals and vessels, such as the gastrointestinal urinal vaginal and vascular tracts are sometimes delivered by a delivery component to a particular site in the body. An example of such device is a balloon catheter on which a balloon expandable stent is positioned.

The use of balloon catheters for dilation of occluded vessels, arteries veins and the like, i.e. angioplasty, has become a standard treatment procedure. This surgical technique typically involves routing a dilation catheter having an inflatable device (balloon) on the distal end thereof through the vascular system to a diseased location within a coronary artery. The inflatable device is then positioned to cover the diseased area of the vessels. A fluid is introduced into the proximal end of the catheter to inflate the inflatable device to a predetermined elevated pressure whereby the diseased area is compressed into the vessel wall. The inflatable device is then deflated and the catheter is removed.

A disadvantage of balloon angioplasty, however, is that the procedure occasionally results in short or long term failure of approximately 60%. To treat recurrent vessel occlusion following balloon angioplasty, implantable endoluminal prostheses, commonly referred to as grafts or stents, has emerged as a means by which to achieve long term vessel patency. Thus, a stent functions as permanent scaffolding to structurally support the vessel wall and thereby maintain coronary luminal patency.

In a typical procedure, stent implantation immediately follows a balloon angioplasty. In order to accommodate presently available stent delivery systems either with a balloon or self-expanding stent, angioplastic dilatation of the lesion must produce a residual lumen large enough to accept the delivery device which surrounds the catheter and passes through an exterior guide catheter. In this regard the apparatus and methods deployed in placing an arterial stent are in many respects similar to those used in an angioplasty procedure.

The stent delivery system normally comprises a stent premounted, such as by crimping, onto a folded expandable balloon at the distal end of a stent delivery catheter. The stent, which is generally fabricated from expandable stainless steel lattice or mesh is normally formed as a substantially cylindrical member. The stent expansion balloon may be formed of polyethylene or other suitable material. The stent delivery system additionally comprises the stent catheter delivery sheath or, more simply, the “delivery sheath” that envelops the stent, delivery catheter, and optionally the balloon and extends substantially the entire length of the delivery catheter.

Once properly positioned relative to the guide catheter, the stent delivery system is extended from the distal end of the guide catheter until the stent spans the previously expanded disease area. Thereafter, the delivery sheath, which is slideable relative to the delivery catheter, balloon and stent, is withdrawn into the guide catheter to expose the stent and, optionally, the balloon. In the case of a balloon-expandable stent assemblies the delivery catheter is then supplied with a pressurized fluid and the fluid expands the balloon. The associated stent is expanded to a desired diameter sufficient to exceed the elastic limit of the stent whereby the stent becomes imbedded in and permanently supports the vessel wall. The balloon is then deflated and it, the stent catheter and guide catheter are withdrawn leaving the expanded stent and an open lumen.

During the stent delivery procedure as the delivery catheter carrying the stent is being maneuvered through the vessel, the stent is subjected to forces which may dislodge the stent from its desired position on the balloon. Also, retention of the stent on the balloon during withdrawal of the delivery sheath prior to implantation may be a problem especially if sheath withdrawal is coupled with subsequent shifting of the stent delivery catheter. Even under the best of circumstances, when a misaligned stent has not yet been deployed and can be successfully retrieved the stent delivery system usually must be withdrawn and the entire procedure repeated using a new assembly. Alternatively, the stent may be disposed so as to partially span or possibly fail to span any portion of the target lesion in which case a supplemental stent placement may be required.

Stent slippage cannot be overcome by simply increasing the crimping force applied when mounting the stent to the folded dilatation balloon. Increased crimping force may result in overcrimping of the stent. Overcrimping may damage the stent, and therefore hinder its proper expansion and implantation, and possibly puncture the balloon.

Other means have been described for retaining a stent in position on a balloon during delivery. For instance, protrusions have been provided on the balloon, or the catheter near to the balloon, having shoulders above and/or below the stent location which bear against the stent when it is subjected to an axial force. U.S. Pat. No. 6,306,144 describes a method to employ differential coating of the catheter and balloon surfaces with different coating compositions to provide slippery areas on the catheter and less slippery coatings or no coating on the balloon surface to provide for retention of a stent on the balloon surface. WO 01/00109 describes using a zwitterionic polymer comprising monomers including a trialkoxysilyl group to provide for retention of a stent on a balloon surface. EP 778012 describes using multiple layers such as a tackifier and de-tackifier layers to produce different levels of coefficient of friction to provide for retention of a stent on a balloon surface.

Disadvantages of these stent retention systems include weakening of the balloon wall, changing the properties of the balloon so that increased pressure is required to inflate the balloon a requirement for additional manufacturing steps adverse effects on the biocompatibility of the system and an increased external diameter of the stent/balloon delivery system.

Thus, there remains a need for improved methods and retention compositions for maintaining proper stent positioning during the stent delivery procedure that are easily applied and remain on the balloon surface.

### SUMMARY OF THE INVENTION

The present invention relates to delivery systems for delivery of a medical device to a location within a body cavity canal or vessel of the body. The system includes the use of a crosslinkable coating composition in both its uncrosslinked and crosslinked forms, to provide improved retention of a surface of the medical device to the surface of a delivery component of the delivery system. The coating composition should improve retention in an amount sufficient to substantially maintain the position of the medical device with respect to the delivery component against the forces the delivery system may encounter during the delivery procedure by increasing the static friction of one surface with respect to the other. The coating composition may be crosslinked to provide a gel matrix that is covalently bound to the surface of one of the components of the system. Desirably, the coating composition of the invention will be covalently bound to a portion of the outer surface of the delivery component.

In another embodiment the composition can be used for a controlled deployment of a medical device from a surface during a surgical procedure.

In another aspect of the invention the coating composition may be coated on the outer surface of a delivery component to increase the static friction of such delivery component in an amount sufficient to substantially maintain the delivery component in a desired position with respect to a surface of a vessel during the treatment portion of a medical procedure. For example, the coating composition may be coated onto a portion of the outer surface of an expandable balloon used in angioplasty. When the expandable balloon is positioned within the body at a desired site and expanded the coated surface will contact a portion of the vessel wall and the balloon shall be substantially maintained in that position within the vessel while the balloon is expanded and until deflation of the balloon begins.

In one aspect of the invention the coating composition is formed on the surface by a process that includes a complexation reaction between carboxylic acid groups and ether groups as described in copending published U.S. Application No. 2002/0041899 A1, which application is assigned to SurModics, Inc., the assignee of the present invention and the disclosure of which is herein incorporated by reference. The complexation reaction serves to both improve the durability and tenacity of the coating and the retention ability of the composition.

As used herein the term “static friction” refers to the ability of one surface to resist displacement relative to a second surface when one surface has forces applied to it, particularly forces encountered by a delivery system as it is navigated through a vessel of the body.

In one embodiment of the invention, the coating composition preferably comprises a polymeric reagent formed by the polymerization of at least two of the following monomers:

a) about 1 to about 30 mole % of a polyether monomer

b) about 1 to about 75 mole % of a carboxylic acid-containing monomer, and

c) an amount of a hydrophilic monomer suitable to bring the composition to 100% (e.g. about 0 to about 93.9 mole % of a hydrophilic monomer).

Optionally, about 0.1 to about 10 mole % of a photoderivatized monomer is also included in the coating composition.

When the polymeric reagent is applied as a coating to the surface of a medical device, noncovalent interactions occur between carboxylic acid groups and ether groups thus contributing to the formation of a gel matrix. The application of UV light provides photochemical attachment to the substrate as well as the formation of covalent, crosslinks within the matrix. The matrix thus formed, provides both improved durability and tenacity of the coating composition.

In another embodiment, the uncrosslinked composition comprises a polymeric reagent formed by the polymerization of the following monomers:

a) methoxy poly(ethylene glycol)methacrylate (“methoxyPEGMA”), as the polyether monomer, in an amount of between about 1 and about 20 mole %,

b) (meth)acrylic acids as the carboxylic acid-containing monomer component, present in an amount of between about 20 and about 50 mole %,

c) photoderivatized monomer, present in an amount of between about 1 to about 7 mole %, and

d) acrylamide monomer, as a hydrophilic monomer, present in an amount sufficient to bring the composition to 100%.

One embodiment of the invention relates to a delivery system comprising a balloon catheter comprising a balloon at or near its distal end, and a stent mounted on the balloon characterized in that at least a portion of the exterior surface of the balloon and/or a portion of the interior surface of the stent that are in contact with each other are provided with the coating composition of the invention to an amount sufficient to increase the static friction between the surfaces. In a preferred embodiment the coating composition is crosslinked to form a gel matrix and to be covalently bound to the surface of the balloon or stent.

### BRIEF DESCRIPTION OF THE DRAWING

FIG. 1 shows a schematic diagram of a device for performing friction measurements by a vertical pinch method described herein.

### DETAILED DESCRIPTION

The present invention provides a medical device delivery system comprising a medical device that will be delivered to a desired location at a site in the body and a delivery component upon which the medical device will be positioned and a coating composition covalently attached to a portion of the surface of the medical device or delivery component or both such that when the medical device is positioned correctly on the delivery component the coating composition will be between contacting surfaces of the medical device and delivery component. The coating composition shall increase the static friction between the two contacting surfaces in an amount sufficient to substantially maintain the position of the medical device on the delivery component against the forces the delivery system may encounter during the delivery procedure. “Substantially” as used herein shall mean that the medical device will not be displaced on the delivery component in an amount that would prevent the medical device from being positioned at the desired site in the body. Desirably the coating composition will increase the static friction of a surface by at least 25%, and preferably by at least 50%.

The coating composition of this invention preferably includes between about 1 and about 30 mole % of a polyether monomer and preferably from about 1 to about 20 mole %. The term “mole %” as used herein will be determined by the molecular weight of the monomer components.

The polyether monomer is preferably of the group of molecules referred to as alkoxy (poly)alkyleneglycol (meth)acrylates. The alkoxy substituents of this group may be selected from the group consisting of methoxy, ethoxy, propoxy, and butoxy. The (poly)alkylene glycol component of the molecule may be selected from the group consisting of (poly)propylene glycol and (poly)ethylene glycol. The (poly)alkylene glycol component preferably has a nominal weight average molecular weight ranging from about 200 g/mole to about 2000 g/mole, and preferably from about 800 g/mole to about 1200 g/mole. Examples of preferred polyether monomers include methoxy PEG methacrylates, PEG methacrylates, and (poly)propylene glycol methacrylates. Such polyether monomers are commercially available, for instance, from Polysciences, Inc., (Warrington, Pa.).

A composition of this invention preferably includes between about 1 to about 75 mole % of a carboxylic acid-containing monomer. Preferred concentrations of the carboxylic acid-containing monomer are between about 20 to about 50 mole %. These monomers can be obtained commercially, for instance, from Sigma-Aldrich, Inc. (St. Louis, Mo.).

Preferred carboxylic acid-containing monomers are selected from carboxyl substituted ethylene compounds, also known as alkenoic acids. Examples of particularly preferred carboxylic acid-containing monomers include acrylic, methacrylic, maleic crotonic, itaconic, and citraconic acid. Most preferred examples of carboxylic acid-containing monomers include acrylic acid and methacrylic acid.

A composition of the present invention preferably includes between about 0.1 and about 10 mole % of a photoderivatized monomer, more preferably between about 1 and about 7 mole %, and most preferably between about 3 and about 5 mole %.

Examples of suitable photoderivatized monomers are ethylenically substituted photoactivatable moieties which include N-[3-(4-benzoylbenzamido)propyl]methacrylamide (“BBA-APMA”), 4(2-acryloxyethoxy)-2-hydroxybenzophenone, 4-methacryloxy-2-hydroxybenzophenone, 4-methacryloxy-2-hydroxybenzophenone, 9-vinyl anthracene, and 9-anthracenylmethyl methacrylate. An example of a preferred photoderivatized monomer is BBA-APMA.

Photoreactive species are defined herein, and preferred species are sufficiently stable to be stored under conditions in which they retain such properties. See, e.g., U.S. Pat. No. 5,002,582, the disclosure of which is incorporated herein by reference. Latent reactive groups can be chosen that are responsive to various portions of the electromagnetic spectrum, with those responsive to ultraviolet and visible portions of the spectrum (referred to herein as “photoreactive”) being particularly preferred.

Photoreactive species respond to specific applied external stimuli to undergo active specie generation with resultant covalent bonding to an adjacent chemical structure, e.g., as provided by the same or a different molecule. Photoreactive species are those groups of atoms in a molecule whose covalent bonds remain unchanged under conditions of storage but upon activation by an external energy source, form covalent bonds with other molecules.

The photoreactive species generate active species such as free radicals and particularly nitrenes, carbenes, and excited states of ketones upon absorption of electromagnetic energy. Photoreactive species can be chosen to be responsive to various portions of the electromagnetic spectrum, and photoreactive species that are responsive to, e.g., ultraviolet and visible portions of the spectrum, are preferred and can be referred to herein occasionally as “photochemical group” or “photogroup.”

The photoreactive species in photoreactive aryl ketones are preferred, such as acetophenone, benzophenone, anthraquinone, quinones, anthrone and anthrone-like heterocycles, i.e., heterocyclic analogs of anthrone such as those having N, O, or S in the 10-position, or their substituted, e.g., ring substituted, derivatives. Examples of preferred aryl ketones include heterocyclic derivatives of anthrone, including acridone, xanthone, and thioxanthone, and their ring substituted derivatives. Particularly preferred are thioxanthone, and its derivatives, having excitation energies greater than about 360 nm.

The functional groups of such ketones are preferred since they are readily capable of undergoing the activation/inactivation/reactivation cycle described herein. Benzophenone is a particularly preferred photoreactive moiety, since it is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (e.g., carbon-hydrogen) is not available for bonding, the ultraviolet light-induced excitation of the benzophenone group is reversible and the molecule returns to ground state energy level upon removal of the energy source Photoactivatable aryl ketones such as benzophenone and acetophenone are of particular importance inasmuch as these groups are subject to multiple reactivation in water and hence provide increased coating efficiency.

The azides constitute a preferred class of photoreactive species and include derivatives based on arylazides (C₆R₅N₃) such as phenyl azide and particularly 4-fluoro-3-nitrophenyl azide, acyl azides (—CO—N₃) such as benzoyl azide and p-methylbenzoyl azide, azido formates (O—CO—N₃) such as ethyl azidoformate, phenyl azidoformate, sulfonyl azides (—SO₂—N₃) such as benzenesulfonyl azide, and phosphoryl azides (RO)₂PON₃ such as diphenyl phosphoryl azide and diethyl phosphoryl azide, Diazo compounds constitute another class of photoreactive species and include derivatives of diazoalkanes (—CHN₂) such as diazomethane and diphenyldiazomethane, diazoketones (—CO—CHN₂) such as diazoacetophenone and 1-trifluoromethyl-1-diazo-2-pentanone, diazoacetates (—O—CO—CHN₂) such as t-butyl diazoacetate and phenyl diazoacetate, and beta-keto-alpha-diazoacetates (—CO—CN₂—CO—O—) such as t-butyl alpha diazoacetoacetate. Other photoreactive species include the diazirines (—CHN₂) such as 3-trifluoromethyl-3-phenyldiazirine, and ketenes (—CH═C═O) such as ketene and diphenylketene.

Upon activation of the photoreactive species, the coating agents are covalently bound to each other and/or to the material surface by covalent bonds through residues of the photoreactive species. Exemplary photoreactive species and their residues upon activation are shown as follows.

| Photoreactive | Group | Residue Functionality |
|---|---|---|
| aryl azides | amine | R—NH—R′ |
| acyl azides | amide | R—CO—NH—R′ |
| azidoformates | carbamate | R—O—CO—NH—R′ |
| sulfonyl azides | sulfonamide | R—SO₂—NH—R′ |
| phosphoryl azides | phosphoramide | (RO)₂PO—NH—R′ |
| diazoalkanes | new C—C bond | |
| diazoketones | new C—C bond and ketone | |
| diazoacetates | new C—C bond and ester | |
| beta-keto-alpha- | new C—C bond and beta- | |
| diazoacetates | ketoester | |
| aliphatic azo | new C—C bond | |
| diazirines | new C—C bond | |
| ketenes | new C—C bond | |
| photoactivated | new C—C bond and alcohol | |
| ketones | | |

The coating agents of the present invention can be applied to any surface having carbon-hydrogen bonds with which the photoreactive species can react to immobilize the coating agents to surfaces.

In another embodiment of the invention it is possible to use a coating composition covalently coupled to the surface without the use of a latent reactive (e.g. photoreactive) group. For instance the surface of the material to be coated can be provided with thermochemically reactive groups which can be used to immobilize polymers containing other thermochemically reactive groups comprising activated esters (e.g. N-oxysuccinimide (“NOS”) epoxide, azlactone, activated hydroxyl, maleimide, alkyl halides, aldehydes, isocyanate or isothiocyanate). For example, a surface may be treated with an ammonia plasma to introduce reactive amines on the surface of the material (e.g. plastic). If the surface is then treated with a polymer having thermochemically reactive groups (e.g. alkyl halide), the polymer can be immobilized through its thermochemical group (alkyl halide) with the corresponding amino groups on the surface. As is known in the art, the reverse procedure can be utilized in which amine derivatized polymers can be coupled to surfaces containing epoxides or other complementary thermally reactive groups.

A composition of the present invention includes a suitable hydrophilic monomer component in an amount sufficient to bring the total composition to 100%. Suitable hydrophilic monomers provide an optimal combination of such properties as water solubility and biocompatibility.

Hydrophilic monomers are preferably taken from the group consisting of alkenyl substituted amides. Examples of preferred hydrophilic monomers include acrylamide, N-vinylpyrrolidone, methacrylamide, acrylamido propanesulfonic acid (AMPS), Acrylamide is an example of a particularly preferred hydrophilic monomer.

Such monomers are available commercially from a variety of sources, e.g. Sigma-Aldrich, Inc. (St. Louis, Mo.) and Polysciences, Inc. (Warrington, Pa.).

In one embodiment of the invention, a medicament is incorporated into the coating composition. The medicament coating composition may be used on a surface of one or both components of the delivery system to allow for delivery of the medicament to a desired location. The word “medicament”, as used herein, will refer to a wide range of biologically active materials or drugs that can be incorporated into a coating composition of the present invention. The substances to be incorporated preferably do not chemically interact with the composition during fabrication, or during the release process.

Medicaments useful with this invention include, without limitation, medicaments selected from the group consisting of gene therapy agents selected from therapeutic nucleic acids and nucleic acids encoding therapeutic gene products, antibiotics selected from penicillin, tetracycline, chloramphenicol, minocycline, doxycycline, vancomycin, bacitracin, kanamycin, neomycin, gentamycin, erythromycin and cephalosporins and antiseptics selected from silver sulfadiazine, chlorhexidine, glutaraldehyde, peracetic acid, sodium hypochlorite, phenols, phenolic compounds, iodophor compounds, quaternary ammonium compounds, and chlorine compounds.

The surfaces of the components of the delivery system of the invention may be formed from polymeric, metallic and/or ceramic materials. In addition, supports such as those formed of pyroltic carbon and silylated surfaces of glass, ceramic, or metal are suitable for surface modification. Suitable polymeric materials include, without limitation, polyurethane and its copolymers, silicone and its copolymers, ethylene vinyl acetate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Metallic materials may also be used in components of the delivery system of the invention, the surfaces of which may be coated with the coating composition. Metallic materials include metals and alloys based on titanium (such as nitinol, nickel titanium alloys, thermo-memory alloy materials), stainless steel, tantalum, nickel-chrome, or cobalt-chromium (such those available under the tradenames Elgiloy™ and Phynox™). Metallic materials also include clad composite filaments, such as those disclosed in WO 94/16646. Examples of ceramic materials include ceramics of alumina and glass-ceramics such as those available under the tradename Macor™.

Optionally, a primer layer(s) may be applied to an inorganic substrate to enhance attachment of polymeric composition(s) to the substrate. Examples of such primer layers include parylene and silane. Parlyene is the generic name for members of a unique polymer (poly-p-xylylene) series several of which are available commercially (e.g., in the form of “Parlyene C”, “Parylene D” and Parylene N,” from Union Carbide).

The components that can be coated with a composition of the present invention include materials that are substantially insoluble in body fluids and that are generally designed and constructed to be placed in or onto the body or to contact fluid of the body. The materials preferably have the physical properties such as strength elasticity permeability and flexibility required to function for the intended purpose; can be purified, fabricated and sterilized easily; will substantially maintain their physical properties and function during the time that they remain implanted in or in contact with the body. Examples of such materials include metals such as titanium alloys, TiNi (shape memory/super elastic) aluminum oxide, platinum platinum alloys, stainless steels, MP35N, elgiloy, haynes 25, stellite, pyrolytic carbon, silver or glassy carbon; polymers such as polyurethanes, polycarbonates, silicone elastomers, polyolefins including polyethylenes or polypropylenes, polyvinyl chlorides, polyethers, polyesters, nylons, polyvinyl pyrrolidones, polyacrylates and polymethacrylates such as polymethylmethacrylate (“PMMA”), n-Butyl cyanoacrylate, polyvinyl alcohols, polyisoprenes, rubber, cellulosics, polyvinylidene fluoride (“PVDF”), polytetrafluoroethylene, ethylene tetrafluoroethylene copolymer (“ETFE”), acrylonitrile butadiene ethylene, polyamide, polyimide, styrene acrylonitrile, and the like; minerals or ceramics such as hydroxyapatite; human or animal protein or tissue such as bones skin, teeth, collagen, laminin, elastin or fibrin; organic materials such as wood, cellulose, or compressed carbon; and other materials such as glass, or the like.

Components of the delivery system made using these materials can be coated or remain uncoated and derivatized or remain underivatized. Medical devices with which a delivery component may be used to position the medical device with which the composition can be used include, but are not limited to, surgical implants prostheses, and any artificial part or device which replaces or augments a part of a living body or comes into contact with bodily fluids, particularly blood, and which is positioned by navigating the medical device through a body vessel channel or canal. As used herein the term “vessel” shall mean any vessel, channel or canal of the body.

Examples of such delivery systems include balloon expandable stent delivery system and self expanding stent delivery systems. The stents may be uncoated or coated with a drug delivery coating such as any such coatings known in the art.

To prepare a delivery system of the invention generally, a solution of the copolymer is prepared at a concentration of about 1% to a concentration of about 20% in water or an aqueous buffer solution. Depending on the surface being coated, an organic solvent such as isopropyl alcohol (“IPA”) can be included in the solution at concentrations varying from about 0 to about 90%. The delivery component or surface to be coated can be dipped into the copolymer solution, or, alternatively, the copolymer solution can be applied to the surface of the component by spraying or the like. At this point, the component can be airs dried to evaporate the solvent or can proceed to the illumination step without drying. The component can be rotated and illuminated with UV light for 30 seconds-to about 10 minutes, or more preferably 30 seconds to 5 minutes, to insure an even coat of the coating. This process can be repeated multiple times to attain the desired coating thickness. Coating thicknesses can be evaluated using scanning electron microscopy (SEM) in both the dry and hydrated forms. The difference in thickness between the dry and the hydrated condition is not generally significant. The thickness of the coating should be sufficient to provide mechanical strength to improve retention of the medical device but not so great as to interfere with the operation of the delivery system. For example, when the delivery system comprises a balloon catheter and a stent, the coating composition should not increase the external diameter of the system by an unacceptable amount. Also, the thickness should not be so great as to increase the pressure at which the balloon deploys the stent.

The amount of increase in the static friction between the two contacting surfaces of the delivery assembly may be determined by polymer and/or solvent selection. Desirably coating a surface of a delivery system with a composition of the invention the static friction between the two contacting surfaces shall be increased by at least 25% over that of an uncoated surface and more desirably increased by at least 50% over that of an uncoated surface. Desirably, the static friction will be increased to obtain improved retention of the medical device on the delivery component by the desired amount (an amount sufficient to substantially maintain the position of the medical device on the delivery component) still allow the medical device to be released from the delivery component once it is placed at the desired location without substantially displacing the medical device from its position.

When medicament is incorporated into the matrix it is done so either by mixing the medicament into the copolymer or incorporating it after the matrix itself has been coated onto the surface of the desired component. Generally a solution of medicament or medicaments is prepared and the matrix-coated device is soaked in the solution. Medicament is absorbed into the matrix from the solution. Various solvents can be used to form the medicament solution as the amount of medicament absorbed by the matrix can be controlled by the solvent solution. Likewise, the pH and/or the ionic strength of the medicament solution can be adjusted to control the degree of medicament absorption by the matrix. After soaking in medicament solution for a period of time, the medical device is removed and air dried.

Another embodiment of the invention relates to a process of producing the delivery system of the invention by coating a portion of the delivery component and/or a portion of the medical device of the system. Such coating methods include, for example, dipping, spraying, brushing, knife coating, and roller coating. The coated surface(s) are then optionally subjected to UV light to cause crosslinking and covalent binding of the composition to the surface. Where the medical device is a stent it is typically positioned on the delivery component after the coating is applied to the delivery component and after the matrix is formed. However, the order of application of the coating and formation of the crosslinked matrix may vary depending on the delivery system and the components thereof.

In the embodiment of the invention wherein the delivery system comprises a balloon catheter and expandable stent, the stent may be crimped onto the catheter after the coating composition is applied.

Other uses of the coating composition of the invention will be apparent to a person skilled in the art. For example, the coating composition can be use with both coated and noncoated stents. It may be used as a tactile depth or positioning system for delivery systems wherein a catheter or wire is advanced through another catheter until a point of resistance on the tip or other selected area is reached. The coating composition could be placed within the catheter to create the point of resistance. Similarly, the coating composition on the surface of a catheter and/or guidewire or other delivery component used to place anastomosis devices and coils within a vessel.

The invention will be further described with reference to the following non-limiting Examples. It will be apparent to those skilled in the art that many changes can be made in the embodiments described in the Examples without departing from the scope of the present invention. Thus the scope of the present invention should not be limited to the embodiments described in this application, but only by the embodiments described by the language of the claims and the equivalents of those embodiments.

### EXAMPLES

### Preparative Example 1

### Preparation of 4- Benzoylbenzoyl Chloride (BBA-C1) (Compound I)

4-Benzoylbenzoic acid (BBA), 1.0 kg (4.42 moles), was added to a dry 5 liter Morton flask equipped with reflux condenser and overhead stirrer, followed by the addition of 645 ml (8.84 moles) of thionyl chloride and 725 ml of toluene. Dimethylformamide, 3.5 ml, was then added and the mixture was heated at reflux for 4 hours. After cooling the solvents were removed under reduced pressure and the residual thionyl chloride was removed by three evaporations using 3×500 ml of toluene. The product was recrystallized from 1:4 toluene:hexane to give 988 g (91% yield) after drying in a vacuum oven. Product melting point was 92-94° C. Nuclear magnetic resonance (“NMR”) analysis at 80 MHz (¹H NMR (CDCl₃)) was consistent with the desired product: aromatic protons 7.20-8.25 (m, 9H). All chemical shift values are in ppm downfield from a tetramethylsilane internal standard. The final compound (Compound I shown below) was stored for use in the preparation of a monomer used in the synthesis of photoactivatable polymers as described, for instance, in Preparative Example 3.

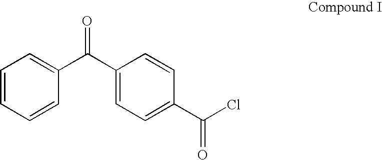

### Preparative Example 2

### Preparation of N-(3-Aminopropyl)methacrylamide Hydrochloride (APMA) (Compound II)

A solution of 1,3-diaminopropane, 1910 g (25.77 moles), in 1000 ml of CH₂Cl₂ was added to a 12 liter Morton flask and cooled on an ice bath. A solution of t-butyl phenyl carbonates 1000 g (5.15 moles), in 250 ml of CH₂Cl₂ was then added dropwise at a rate which kept the reaction temperature below 15° C. Following the addition, the mixture was warmed to room temperature (approx. 25° C.) and stirred 2 hours. The reaction mixture was diluted with 900 ml of CH₂Cl₂ and 500 g of ice, followed by the slow addition of 2500 ml of 2.2 N NaOH. After testing to insure the solution was basic, the product was transferred to a separatory funnel and the organic layer was removed and set aside as extract #1. The aqueous was then extracted with 3×1250 ml of CH₂Cl₂, keeping each extraction as a separate fraction. The four organic extracts were then washed successively with a single 1250 ml portion of 0.6 N NaOH beginning with fraction #1 and proceeding through fraction #4. This wash procedure was repeated a second time with a fresh 1250 ml portion of 0.6 N NaOH. The organic extracts were then combined and dried over Na₂SO₄. Filtration and evaporation of solvent to a constant weight gave 825 g of N-mono-t-BOC-1,3-diaminopropane which was used without further purification.

A solution of methacrylic anhydride, 806 g (5.23 moles), in 1020 ml of CHCl₃ was placed in a 12 liter Morton flask equipped with overhead stirrer and cooled on an ice bath. Phenothiazine, 60 mg, was added as an inhibitor, followed by the dropwise addition of N-mono t-BOC-1,3-diaminopropane, 825 g (4.73 moles), in 825 ml of CHCl₃. The rate of addition was controlled to keep the reaction temperature below 10° C. at all times. After the addition was complete, the ice bath was removed and the mixture was left to stir overnight. The product was diluted with 2400 ml of water and transferred to a separatory funnel. After thorough mixing, the aqueous layer was removed and the organic layer was washed with 2400 ml of 2 N NaOH, insuring that the aqueous layer was basic The organic layer was then dried over Na₂SO₄ and filtered to remove the drying agent. A portion of the CHCl₃ solvent was removed under reduced pressure until the combined weight of the product and solvent was approximately 3000 g. The desired product was then precipitated by slow addition of 11.0 liters of hexane to the stirred CHCl₃ solution, followed by overnight storage at 4° C. The product was isolated by filtration and the solid was rinsed twice with a solvent combination of 900 ml of hexane and 150 ml of CHCl₃. Thorough drying of the solid gave 900 g of N-[N′-(t-butyloxycarbonyl)-3-aminopropyl]-methacrylamide, m.p. 85.8° C. by differential scanning calorimetry (“DSC”) Analysis on an NMR spectrometer was consistent with the desired product: ¹H NMR (CDCl₃) amide NH's 6.30-6.80, 4.55-5.10 (m, 2H), vinyl protons 5.65, 5.20 (m, 2H), methylenes adjacent to N, 2.90-3.45 (m, 4H), methyl 1.95 (m, 3H), remaining methylene 1.50-1.90 (m, 2H), and t-butyl 1.40 (s, 9H).

A 3-neck, 2 liter round bottom flask was equipped with an overhead stirrer and gas sparge tube. Methanol, 700 ml, was added to the flask and cooled on an ice bath. While stirring, HCl gas was bubbled into the solvent at a rate of approximately 5 liters/minute for a total of 40 minutes. The molarity of the final HCl/MeOH solution was determined to be 8.5 M by titration with 1 N NaOH using phenolphthalein as an indicator. The N-[N′-(t-butyloxycarbonyl)-3-aminopropyl]methacrylamide 900 g (3.71 moles), was added to a 5 liter Morton flask equipped with an overhead stirrer and gas outlet adapter, followed by the addition of 1150 ml of methanol solvent. Some solids remained in the flask with this solvent volume, Phenothiazine, 30 mg, was added as an inhibitor, followed by the addition of 655 ml (5.57 moles) of the 8.5 M HCl/MeOH solution. The solids slowly dissolved with the evolution of gas but the reaction was not exothermic. The mixture was stirred overnight at room temperature to insure complete reaction. Any solids were then removed by filtration and an additional 30 mg of phenothiazine were added. The solvent was then stripped tinder reduced pressure and the resulting solid residue was azeotroped with 3×1000 ml of isopropanol with evaporation under reduced pressure. Finally, the product was dissolved in 2000 ml of refluxing isopropanol and 4000 ml of ethyl acetate were added slowly with stirring. The mixture was allowed to cool slowly and was stored at 4° C. overnight. Compound II was isolated by filtration and was dried to constant weight, giving a yield of 630 g with a melting point of 124.7° C. by DSC. Analysis on an NMR spectrometer was consistent with the desired product: ¹H NMR (D₂O) vinyl protons 5.60, 5.30 (m, 2H), methylene adjacent to amide N, 3.30 (t, 2H), methylene adjacent to amine N, 2.95 (t, 2H), methyl 1.90 (m, 3H), and remaining methylene 1.65-2.10 (m, 2H). The final compound (Compound II shown below) was stored for use in the preparation of a monomer used in the synthesis of photoactivatable polymers as described, for instance, in Preparative Example 3.

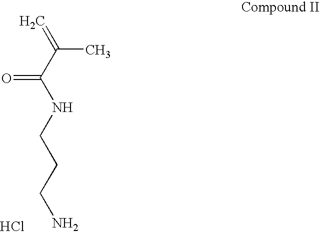

### Preparative Example 3

### Preparation of N-[3-(4-Benzoylbenzamido)propyl]methacrylamide (BBA-APMA) (Compound III)

Compound II 120 g (0.672 moles) prepared according to the general method described in Preparative Example 29 was added to a dry 2 liter three-neck round bottom flask equipped with an overhead stirrer. Phenothiazine, 23-25 mg, was added as an inhibitor, followed by 800 ml of chloroform. The suspension was cooled below 10° C. on an ice bath and 172.5 g (0.705 moles) of Compound I, prepared according to the method described in Example 19 were added as a solid. Triethylamine, 207 ml (1.485 moles)₉ in 50 ml of chloroform was then added dropwise over a 1-1.5 hour time period. The ice bath was removed and stirring at ambient temperature was continued for 2.5 hours. The product was then washed with 600 ml of 0.3 N HCl and 2×300 ml of 0.07 N HCl. After drying over sodium sulfates the chloroform was removed under reduced pressure and the product was recrystallized twice from 4:1 toluene:chloroform using 23-25 mg of phenothiazine in each recrystallization to prevent polymerization. Typical yields of Compound III were 90% with a melting point of 147-151° C. Analysis on an NMR spectrometer was consistent with the desired product: ¹H NMR (CDCl₃) aromatic protons 7.20-7.95 (m, 9H), amide NH 6.55 (broad t, 1H), vinyl protons 5.65, 5.25 (m, 2H), methylenes adjacent to amide N's 3.20-3.60 (m, 4H), methyl 1.95 (s, 3H), and remaining methylene 1.50-2.00 (m, 2H). The final compound (Compound III shown below) was stored for use in the synthesis of photoactivatable polymers as described in Preparative Examples 4 and 5.

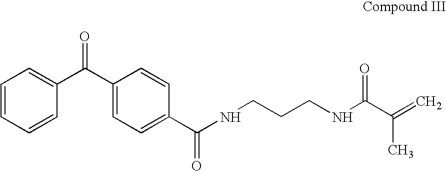

### Example 4

### Preparation of Polyacrylamide-(36%)co-Methacrylic acid (MA)-(10%)co-Methoxy PEG1000MA-(4%)co-BBA-APMA (Compound IV)

Acrylamide, 37.3 g (0.52 mole), and BBA-APMA (Compound III), 14.7 g (0.04 moles), were dissolved in dimethylsulfoxide (“DMSO”), followed by methoxypolyethyleneglycol 1000 monomethacrylate (methoxy PEG 1000 MA) 115.5 g (0.11 mole), methacrylic acid, 32.5 g (0.38 mole), 2,2′-azobis(2-methylbutyronitrile) (Vazo® 679 manufactured by E.I. DuPont de Nemours & Company), 2.5 g (0.01 mole). The solution was deoxygenated with a nitrogen sparge for 10 minutes at 60° C., then blanketed with nitrogen and heated overnight at 60° C. The resulting product was diafiltered against deionized water using a 10,000 molecular weight cutoff cassette, then lyophilized to give 190 g of polymer. The resultant polymer was identified as acrylamide-co-methacrylic acid-co-methoxy PEG 1000 MA-co-BBA-APMA having the following general structure (Compound IV).

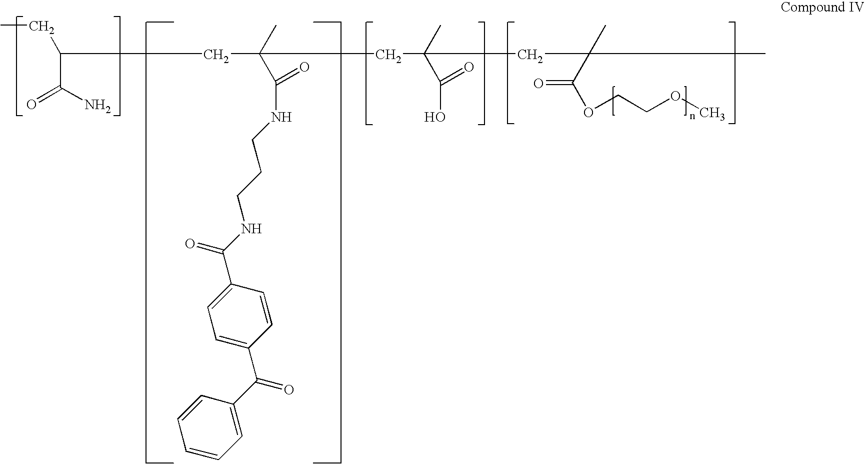

### Example 5

### Preparation of Various Analogs of Compound IV

A series of polymers of the general formula of Compound IV were synthesized as generally described in Example 4. The mole percent of acrylamide, methoxy PEG 1000 monomethacrylate, and methacrylic acid were varied while the mole percent of the BBA-APMA (Compound III) was held constant at four mole percent. The ratios of the other groups to carbonyl groups in the various polymers were calculated assuming each mole of the methoxy PEG 1000 monomethacrylate contained 23 ether groups. A list of the various polymers prepared and the composition of the various polymers are listed below.

The following compounds were synthesized in a manner analogous to that described above with respect to Compound IV.

| | |
|---|---|
| Compound IV | 4% BBA-APMA, 10% methoxy PEG 1000 MA, 36% |
| | methacrylic acid, 50% acrylamide |
| Compound V | 4% BBA-APMA, 2% methoxy PEG 1000 MA, 28% |
| | methacrylic acid, 66% acrylamide |
| Compound VI | 4% BBA-APMA, 26% methoxy PEG 1000 MA, 28% |
| | methacrylic acid, 42% acrylamide |
| Compound VII | 4% BBA-APMA, 14% methoxy PEG 1000 MA, 40% |
| | methacrylic acid, 42% acrylamide |
| Compound VIII | 4% BBA-APMA, 10% methoxy PEG 1000 MA, 86% |
| | acrylamide |
| Compound IX | 4% BBA-APMA, 46% methacrylic acid, 50% |
| | acrylamide |

Table 1 also shows the composition of the polymers.

TABLE 1
Composition of polymers prior to making coating solutions.

| | | Mole % | Mole % |
|---|---|---|---|
| | Mole % | MeO-PEG | Methaerylic |
| Compound | Acrylamide | 1000 | Acid |
| IV | 50 | 10 | 36 |
| V | 66 | 2 | 28 |
| VI | 42 | 26 | 28 |
| VII | 42 | 14 | 40 |
| VIII | 86 | 10 | 0 |
| IX | 50 | 0 | 46 |

### Example 6

### Stent Retention

We demonstrated the stent retention coating ability of the coating composition of this invention by coating the balloon of a stent delivery catheter assembly. Polymer coatings for Compounds IV-IX were applied to the balloon of a stent delivery catheter assembly using a dip coating process (described below) and cured using ELC 4000 lamps (Electro-lite Corp, Danbury, Conn.), approximately 40 cm apart, and containing 400 watt mercury vapor bulbs which put out 1.5 mW/sq. cm from 330-340 nm. After the coating process a stainless steel stent was crimped onto the balloon using well-known methods.

Polymer coating solutions containing Compounds IV-VIII were made by mixing 50 mg/ml of each compound in a 50/50 IPA and deionized water solution. For the solution comprising Compound IX, a polymer coating solution was made by mixing 25 mg/ml of the polymer in a 50/50 IPA and deionized water solution. The balloon of a stent delivery catheter assembly was coated by the following dip coating process. The balloon were dipped into the polymer coating solution at a rate 2.0 cm/sec. and allowed to soak in the solution for 5 seconds. The balloon was withdrawn from the solution at a rate of 1.0 cm/sec. The balloon was air-dried for 10 minutes. After air-drying the balloon was exposed to the previously described UV light system for 3 minutes.

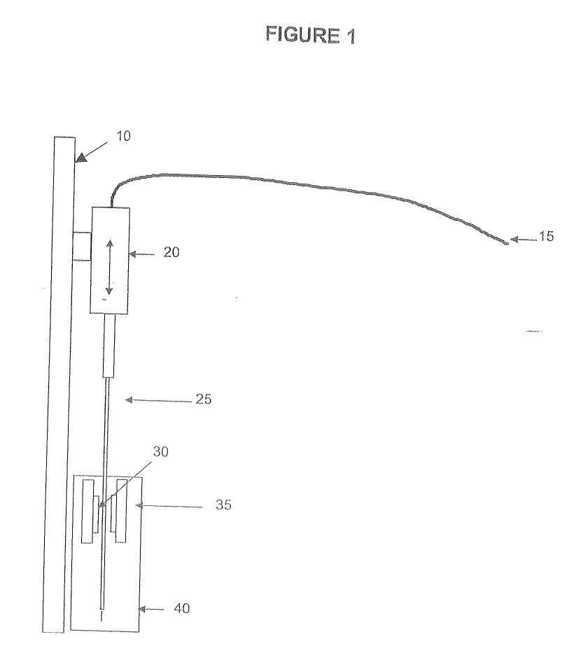

After coating, the balloon stent delivery catheter assembly was evaluated for increased static friction between the balloon and stent surfaces (peak force to break free) using a Vertical Pinch Tester shown in FIG. 1 and the method described below. As shown in FIG. 1 a force gauge 20 is attached to a motion control rail 10 (vertical motion) whereby the amount of force required for the balloon to break free of the stent surface is measured. The output force is measured by measuring means 15 and recorded by recording means, not shown, which is typically a PC.

The results were obtained by inserting the crimped stent and balloon catheter assembly 25 between the two jaws 35 of the pinch tester. Silicone pads 30 are attached to the inside of the jaws 35. The pinch tester jaws are immersed in a cylinder of water or saline 40.

In this experiment the proximal end of the catheter was affixed to a Chatillon force gauge 20 (Model DFGS-2, AMETEK, Paoli, Pa.) and attached to the motion control rail 10. The jaws 35 of the pinch tester were closed as the stent balloon catheter assembly 25 was pulled in a vertical direction and opened when the assembly was returned to the original position. A calibrated pinch force of 500 grams measured with a strain gauge meter 15 (Model DP25-S, Omega Engineering INC. Stamford Conn.), was applied to each balloon stent assembly. The static friction was determined for 3 cycles as the balloon traveled 3 cm at a 0.1 cm/s travel speed. The force (grams) was recorded as the stent was pulled from the balloon stent catheter assembly 25, as measured with the strain gauge meter. The maximum or peak static friction force to dislodge the balloon from the stent is summarized in Table 2.

TABLE 2
Friction Evaluation on Balloons for Stent Retention

| | Static friction (n = 3) − | |
|---|---|---|
| Compound | grams | % difference from Uncoated |
| IV | 152 | 62% |
| V | 196 | 109% |
| VI | 132 | 40% |
| VII | 166 | 77% |
| VIII | 189 | 101% |
| IX | 118 | 26% |
| Uncoated | 94 | 0% |

### Example 7

### Preparation of 1-[(chloroacetyl)oxy]succinimide (Cl-Acetyl-NOS) (Compound X)

Chloroacetic acid, 5.0 g (52.9 mmole), and N-hydroxysuccinimide (NHS), 6.39 g (55.6 mmole) were placed in a flask with a magnetic stir bar and dioxane (1,4-dioxane, 15 ml). Dicyclohexylcarbodiimide (DCC), 12.0 g (58.2 mmole), was dissolved in dioxane (10 ml). The DCC solution was added to the chloroacetic acid/NHS solution 1 ml at a time over 20 minutes with occasional cooling. After the DCC solution was added the flask was rinsed with dioxane (5 ml) and added to the reaction. The reaction flask was stirred in an ice bath which was allowed to come to room temperature over night. The reaction mixture was filtered to remove dicyclohexylurea (DCU). The DCU was washed once with dioxane (5 ml), and a second time with dioxane (10 ml). A 0.2 ml sample was evaporated and dissolved in CDCl₃, Analysis on a 400 MHz NMR spectrometer was consistent with the desired product: ¹H NMR (CDCl₃) methylene adjacent to chlorine 4.38 (s, 2H), and methylenes of the succinimide ring 2.87 (s, 4H).

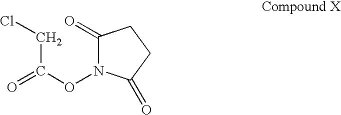

### Example 8

### Preparation of N-{3-[(chloroacetyl)amino]propyl}methacrylamide (Cl-Acetyl-APMA) (Compound XI)

APMA (Compound III) 8.84 g (49.5 mmole), prepared according to the general method described in Preparative Example 3, was placed in a flask. The dioxane solution of Compound X, prepared according to the general method described in Example 7, ˜44 ml (52.9 mmole) was added to the flask containing Compound III. To the mixture was added triethylamine, 6.9 ml (49.5 mmole). The reaction was stirred for 2 hours. The reaction mixture was placed in 550 ml of water containing con. HCl, 2.75 ml (33 mmole), and extracted with 3×110 ml CHCl₃. The combined CHCl₃ solutions were washed with 110 ml of 0.05 N HCl. The volatiles were removed on a rotary evaporator to give 7.17 g of crude Compound XI. The crude product was purified using a silica gel column 1⅝″ diameter×99″ long. The column was eluted with 65×38 ml fractions of acetone/CHCl₃-20/80. Fractions 23 to 60 were combined and evaporated to give 6.38 g Compound XI (59% yield), Analysis on a 400 MHz NMR spectrometer was consistent with the desired product: ¹H NMR (CDCl₃) the amide protons 7.3 and 6.66 (broad, 2H), vinyl protons 5.77, 5.36 (m, 2H), methylene adjacent to chlorine 4.07 (s, 2H), methylenes adjacent to amide N's 3.34-3.40 (m, 4H), methyl 1.99 (s, 3H), and the central methylene 1.69-1.75 (m, 2H).

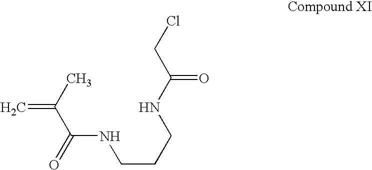

### Example 9

### Preparation of polyacrylamide-(36%)co-Methacrylic acid-(10%)co-Methoxy PEG1000MA-(4%) co-Cl-acetyl-APMA (Compound XII)

Compound XII is made by placing acrylamide, 37.0 g (521 mmole); Cl-acetyl-APMA (Compound XI) 9.1 g (42 mmole); methoxy PEG 1000 MA, 111.5 g (104 mmole) methacrylic acid, 32.3 g (375 mmole); and 2,2′-azobis(2-methylbutyronitrile) (“Vazo® 679 manufactured by E.I. DuPont de Nemours and Company”), 2.5 g (13 mmole) in DMSO 850 ml. The solution is then sparged with nitrogen for 10 minutes, and heated to 60° C. overnight under a nitrogen blanket. The resulting product is diafiltered against deionized water using a 10,000 molecular weight cutoff cassette, and lyophilized. The product Compound XII is a solid with an expected weight of 190 g.

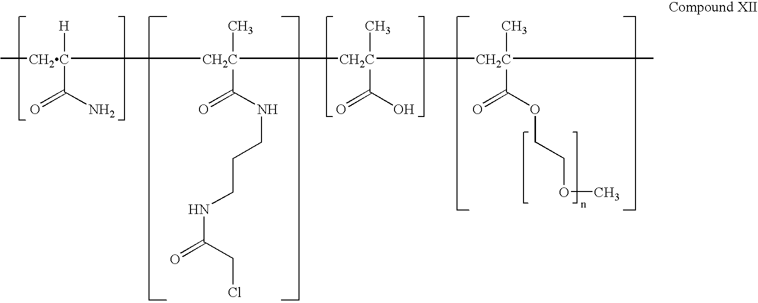

### Example 10

### Coating of a Stainless Steel Flat with Compound XI

A metal flat (0.0254 cm×0.5 cm×2.5 cm) of stainless steel (316L, Goodfellow Cambridge Ltd., Huntingdon, England) is placed in a small vessel containing approximately 50 ml of isopropyl alcohol (IPA) and sonicated in IPA for 20-minutes at 50-60 hz in a Branson 5210RDTH (Branson Ultrasonic Corp., Danbury, Conn.). Next, the metal flat is wiped with IPA followed by sonication for 20 minutes in a 10% Valtron SP2200 (Valtech Corp., Pottstown, Pa.) solution in hot tap water (approx. 50° C.). The metal flat is rinsed in hot tap water to remove most of the detergent, then sonicated for 2 minutes in hot tap water. The metal flat is rinsed in deionized water followed by sonication for 2-minutes in deionized water. As a final preparative step, the metal flat is sonicated for 2-minutes in IPA and followed by drying at room temperature for approximately 2-5 minutes.

The stainless steel metal flat is dipped into a solution of 3-aminopropyltrimethoxysilane (S1A0611.0 Gelest. Inc., Tullytown, Pa.) in acetonitrile/THF and allowed to soak for three minutes. The silane coated metal flat is removed from the silane solution at the rate of 0.05 cm/sec. The silane coated metal is dried at room temperature for at least five minutes followed by further drying in an oven for 15 to 20 minutes at 110° C.

After the silane pretreatment, the flats are allowed to react in a solution of Compound XII. A solution of Compound XII is prepared at a concentration of 50 mg/ml in 50/50 (IPA) and deionized (DI) water. The flats are soaked in 50 mls of 50/50 IPA/DI water overnight at room temperature. The flats are removed from the polymer solutions washed with DI water and allowed to thoroughly dry before evaluation.

### Example 11

### Coating of Compound XII on an Amine Derivatized Surface

A polymer surface is derivatized by plasma treatment using a 3:1 mixture of methane and ammonia gases. (See, e.g., the general method described in U.S. Pat. No. 5,643,580, the disclosure of which is herein incorporated by reference). A mixture of methane (490 Standard Centimeter Cube per Minute) and ammonia (161 Standard Centimeter Cube per Minute) are introduced into the plasma chamber along with the polymer part to be coated. The gases are maintained at a pressure of 0.2-0.3 torr) and a 300-500 watt glow discharge is established within the chamber. The sample is treated for a total of 35 minutes under these conditions. Formation of an amine derivatized surface is verified by a reduction in the water contact angle compared to the uncoated surface.

The amine derivatized surface is incubated with a solution of Compound XII prepared at a concentration of 50 mg/ml in 50/50 IPA/DI water. The surface is allowed to soak in the polymer solution overnight at room temperature. The surface is removed from coating solution washed with DI water and thoroughly dried at room temperature before use.

Using the methods described in Examples 7-11, the coating composition of the invention may be covalently bound to a desired surface thermochemically.

1-43. (canceled)

44. A method of increasing the static friction of a portion of a surface of a delivery system comprising:
providing a coating composition comprising a polymeric reagent, the polymeric reagent being formed by the polymerization of the following monomers:
a) about 1 to about 30 mole % of a polyether monomer,
b) about 1 to about 75 mole % of a carboxylic acid-containing monomer, and
c) an amount of a hydrophilic monomer suitable to bring the composition to 100%;
applying at the coating composition onto at least a portion of a surface of the delivery component under conditions suitable to covalently bind the polymeric reagent to the surface in an amount sufficient to increase the static friction of the surface of the delivery component in an amount sufficient to substantially maintain contact of the coated surface of the delivery component with another surface against forces asserted on the system.

45. A method according to claim 44 wherein the polyether monomer comprises an alkoxy poly(alkyleneglycol) methacrylate.

46. A method according to claim 45 wherein the alkoxy group is selected from the group consisting of methoxy ethoxy, propoxy, and butoxy.

47. A method according to claim 45 wherein the polyalkylene glycol component of the alkoxy poly(alkyleneglycol) methacrylate is selected from the group consisting of polypropylene glycol and polyethylene glycol.

48. A method according to claim 47 wherein the polyalkylene glycol has a nominal weight average molecular weight ranging from about 200 g/mole to about 2000 g/mole.

49. A method according to claim 48 wherein the polyether monomer is selected from the group consisting essentially of methoxy (poly)ethylene glycol methacrylates, (poly)ethylene glycol methacrylates, and (poly)propylene glycol methacrylates.

50. A method according to claim 44 wherein the polyether monomer is present in an amount of between about 1 and about 20 mole %.

51. A method according to claim 44 wherein the carboxylic acid-containing monomer is selected from carboxyl substituted ethylene compounds.

52. A method according to claim 51 wherein the carboxyl acid-containing monomer is selected from acrylic, methacrylic, maleic, crotonic, itaconic, and citraconic acid.

53. A method according to claim 50 wherein the concentration of the carboxylic acid-containing monomer is between about 20 to about 50 mole %.

54. A method according to claim 53 wherein the carboxylic-acid containing monomer comprises (meth)acrylic acid.

55. A method according to claim 53 wherein the concentration of the carboxylic acid-containing monomer is between about 20 to about 50 mole % and the carboxylic acid containing monomer comprises (meth)acrylic acid.

56. A method according to claim 44 wherein the photoderivatized monomer is selected from the group consisting of N-[3-(4-benzoylbenzamido)propyl]met-hacrylamide, 9-vinyl anthracene, and 9-anthracenylmethyl methacrylate.

57. A method according to claim 56 wherein the photoderivatized monomer is present in an amount of between about 1 to about 7 mole %.

58. A method according to claim 44 wherein the hydrophilic monomer comprises an alkenyl substituted amide.

59. A method according to claim 58 wherein the hydrophilic monomer is selected from the group consisting of acrylamide, N-vinylpyrrolidone, methacrylamide, and acrylamido propanesulfonic acid (AMPS).

60. A method according to claim 44 wherein the coating composition increases the static friction of the surface by at least 25%.

61. A method according to claim 44 wherein the coating composition increases the static friction of the surface by at least 50%.

62. A method according to claim 44 wherein a medicament is incorporated into the coating composition.

63-65. (canceled)

66. A method of preparing a delivery system for delivering a medical device to a desired location in the body comprising:
providing a coating composition comprising a polymeric reagent, the polymeric reagent being formed by the polymerization of the following monomers:
a) about 1 to about 30 mole % of a polyether monomer,
b) about 1 to about 75 mole % of a carboxylic acid-containing monomer, and
c) an amount of a hydrophilic monomer suitable to bring the composition to 100%;
applying at the coating composition onto at least a portion of a surface of the delivery component that is in contact with a portion of the medical device, a portion of a surface of the medical device in contact with a portion of the surface of the delivery surface or to both surfaces under conditions suitable to covalently bind the polymeric reagent to such surface in an amount sufficient to increase the static friction of the surface in an amount sufficient to substantially maintain contact of the surface of the delivery component with the surface of the medical device against forces asserted on the system as the system is navigated through a vessel of the body; and
placing the medical device on the delivery component so that the coated surface is located between the two contacting surfaces.

67. A method according to claim 66 wherein the polyether monomer comprises an alkoxy poly(alkyleneglycol) methacrylate.

68. A method according to claim 67 wherein the alkoxy group is selected from the group consisting of methoxy, ethoxy, propoxy, and butoxy.

69. A method according to claim 67 wherein the polyalkylene glycol component of the alkoxy poly(alkyleneglycol) methacrylate is selected from the group consisting of polypropylene glycol and polyethylene glycol.

70. A method according to claim 69 wherein the polyalkylene glycol has a nominal weight average molecular weight ranging from about 200 g/mole to about 2000 g/mole.

71. A method according to claim 70 wherein the polyether monomer is selected from the group consisting essentially of methoxy (poly)ethylene glycol methacrylates, (poly)ethylene glycol methacrylates and (poly)propylene glycol methacrylates.

72. A method according to claim 66 wherein the polyether monomer is present in an amount of between about 1 and about 20 mole %.

73. A method according to claim 66 wherein the carboxylic acid-containing monomer is selected from carboxyl substituted ethylene compounds.

74. A method according to claim 73 wherein the carboxyl acid-containing monomer is selected from acrylic, methacrylic maleic, crotonic itaconic and citraconic acid.

75. A method according to claim 73 wherein the concentration of the carboxylic acid-containing monomer is between about 20 to about 50 mole %.

76. A method according to claim 75 wherein the carboxylic-acid containing monomer comprises (meth)acrylic acid.

77. A method according to claim 74 wherein the concentration of the carboxylic acid-containing monomer is between about 20 to about 50 mole % and the carboxylic acid containing monomer comprises (meth)acrylic acid.

78. A method according to claim 66 wherein the photoderivatized monomer is selected from the group consisting of N-[3-(4-benzoylbenzamido)propyl]methacrylamide, 9-vinyl anthracene, and 9-anthracenylmethyl methacrylate.

79. A method according to claim 78 wherein the photoderivatized monomer is present in an amount of between about 1 to about 7 mole %.

80. A method according to claim 66 wherein the hydrophilic monomer comprises an alkenyl substituted amide.

81. A method according to claim 80 wherein the hydrophilic monomer is selected from the group consisting of acrylamide, N-vinylpyrrolidone, methacrylamide, and acrylamido propanesulfonic acid (AMPS).

82. A method according to claim 66 wherein the coating composition increases the static friction of the surface by at least 25%.

83. A method according to claim 66 wherein the coating composition increases the static friction of the surface by at least 50%.

84. A method according to claim 66 wherein a medicament is incorporated into the coating composition.

85. A method according to claim 66 wherein the medical device is coated with a drug delivery coating.

86. A method according to claim 66 wherein the medical device is a stent.

87. A method according to claim 66 wherein the stent is a self-expanding stent.

88-89. (canceled)

90. A method according to claim 44 wherein the polyether monomer comprises an alkoxy poly(alkyleneglycol) methacrylate, the carboxylic acid-containing monomer is selected from carboxyl substituted ethylene compounds, the photoderivatized monomer is selected from the group consisting of N-[3-(4-benzoylbenzamido)propyl]methacrylamide, 9-vinyl anthracene, and 9-anthlacenylmethyl methacrylate, and the hydrophilic monomer is selected from the group consisting of acrylamide, N-vinylpyrrolidone, methacrylamide, and acrylamido propanesulfonic acid (AMPS).

91. A method according to claim 66 wherein the polyether monomer comprises an alkoxy poly(alkyleneglycol) methacrylate, the carboxylic acid-containing monomer is selected from carboxyl substituted ethylene compounds, the photoderivatized monomer is selected from the group consisting of N-[3-(4-benzoylbenzamido)propyl]methacrylamide, 9-vinyl anthracene, and 9-anthracenylmethyl methacrylate, and the hydrophilic monomer is selected from the group consisting of acrylamide, N-vinylpyrrolidone, methacrylamide, and acrylamido propanesulfonic acid (AMPS).

92. A system according to claim 84 wherein the medicament is selected from the group consisting of gene therapy agents selected from therapeutic nucleic acids and nucleic acids encoding therapeutic gene products, antibiotics selected from penicillin, tetracycline, chloramphenicol, minocycline, doxycycline, vancomycin, bacitracin, kanamycin, neomycin, gentamycin, erythromycin and cephalosporins and antiseptics selected from silver sulfadiazine, chlorhexidine, glutaraldehyde, peracetic acid, sodium hypochlorite, phenols, phenolic compounds, iodophor compounds, quaternary ammonium compounds and chlorine compounds.

93. A method according to claim 84 wherein the medicament is selected from the group consisting of gene therapy agents selected from therapeutic nucleic acids and nucleic acids encoding therapeutic gene products antibiotics selected from penicillin, tetracycline, chloramphenicol, minocycline doxycycline vancomycin, bacitracin kanamycin, neomycin gentamycin, erythromycin and cephalosporins and antiseptics selected from silver sulfadiazine, chlorhexidine, glutaraldehyde, peracetic acid, sodium hypochlorite, phenols, phenolic compounds iodophor compounds, quaternary ammonium compounds, and chlorine compounds.

94-95. (canceled)

96. A coating composition for use in increasing the static friction of a surface of a delivery component of a delivery system in an amount sufficient to increase the static friction so that when a surface of the medical device is in contact with the coating composition and the surface of the delivery component, the static friction is increased in an amount sufficient to maintain the medical device on the delivery component without substantial displacement of the delivery component during navigation of the delivery system though a vessel of the body and wherein the coating composition allows the medical device to be released from the surface of the delivery component once the medical device has been placed at a desired location vessel, the composition comprising a polymeric reagent formed by the polymerization of the following monomers:
a) about 1 to about 30 mole % of a polyether monomer,
b) about 1 to about 75 mole % of a carboxylic acid-containing monomer,
c) an amount of a hydrophilic monomer suitable to bring the composition to 100%.

97-99. (canceled)

100. A method of increasing the static friction of a portion of a surface of a delivery system comprising: providing a crosslinked coating composition comprising a polymeric reagent in the form of a gel matrix, the polymeric reagent being formed by the polymerization of the following monomers:
a) about 1 to about 30 mole % of a polyether monomer
b) about 1 to about 75 mole % of a carboxylic acid-containing monomer and
c) an amount of a hydrophilic monomer suitable to bring the composition to 100%;
applying at the coating composition onto at least a portion of a surface of the delivery component under conditions suitable to covalently bind the polymeric reagent to the surface in an amount sufficient to increase the static friction of the surface of the delivery component in an amount sufficient to substantially maintain contact of the coated surface of the delivery component with another surface against forces asserted on the system.

101. A method of preparing a delivery system for delivering a medical device to a desired location in the body comprising:
providing a cross-linked coating composition comprising a polymeric reagent in the form of a gel matrix, the polymeric reagent being formed by the polymerization of the following monomers:
a) about 1 to about 30 mole % of a polyether monomer,
b) about 1 to about 75 mole % of a carboxylic acid-containing monomer, and
c) an amount of a hydrophilic monomer suitable to bring the composition to 100%;
applying at the coating composition onto at least a portion of a surface of the delivery component that is in contact with a portion of the medical device, a portion of a surface of the medical device in contact with a portion of the surface of the delivery surface or to both surfaces under conditions suitable to covalently bind the polymeric reagent to such surface in an amount sufficient to increase the static friction of the surface in an amount sufficient to substantially maintain contact of the surface of the delivery component with the surface of the medical device against forces asserted on the system as the system is navigated through a vessel of the body; and
placing the medical device on the delivery component so that the coated surface is located between the two contacting surfaces.

102-106. (canceled)
